(19) United States
(12) Patent Application Publication
(10) Pub. No.: US 2009/0172867 A1
(43) Pub. Date: Jul. 9, 2009

(54) FOOT COVERING

(76) Inventors: N. Christian Kopp, Overland Park, KS (US); Russell Davis, Larkspur, CA (US)

Correspondence Address: HOVEY WILLIAMS LLP, 10801 Mastin Blvd., Suite 1000, Overland Park, KS, 66210, US

(21) Appl. No.: 12/046,153

(22) Filed: Mar. 11, 2008

Related U.S. Application Data
(63) Continuation of application No. 10/839,413, filed on May 5, 2004, now abandoned.

(51) Int. Cl. A43B 17/00; A61M 35/00

(52) U.S. Cl. 2/239; 604/293

(57) ABSTRACT

A form-fitting foot covering (10, 34, 44) is provided including a sole portion (14, 36, 46) and a corresponding uppers portion (16, 38, 48) which is designed to be worn in a tightly fitting manner over a user's foot to provide protection and comfort. The inner surface (20) of the foot covering (16, 34, 44) may carry a medicament (22). The foot covering (10, 34, 44) may be integrally formed using a thin, resilient, stretchable resin material such as latex, vinyl or nitrile, and they may be equipped with a laminated resilient gel padding layer (30). Alternately, the uppers portion (38) may be formed of a woven cloth material (40) or a perforate synthetic resin material presenting air holes (52). An optional adhesive (60) may be used to hold the foot covering in place and create a watertight seal.

### RELATED APPLICATION

This is a continuation of application Ser. No. 10/839,413 filed May 5, 2004, the teachings and content of each of which are hereby incorporated by reference herein in its entirety.

### BACKGROUND OF THE INVENTION

1. Field of the Invention

The present invention is broadly concerned with form-fitting foot socks adapted to be worn as comfortable and protective coverings over the feet, lower leg, and/or upper leg of users. More particularly, the invention is concerned with such foot coverings used to create a protective barrier over the foot that may be watertight and may be used to apply medicaments to the skin of the user.

2. Description of the Prior Art

The use of latex or other synthetic resin gloves by medical or food service personnel is well-established. It is known that these glove products inhibit the spread of potentially harmful microorganisms, in order to protect both the wearer and others.

However, there are many other vectors for infection other than through the hands. For example, it is quite common that Athlete's Foot can be spread in locker and shower rooms, owing to the fact that users generally are in their bare feet. This is particularly troublesome if a particular person has extant foot problems or bandages on his feet or ankles.

Protective coverings have previously been provided for leg or arm casts, in order to allow the wearer to shower and bathe. However, these prior products are typically of bag-like construction, meaning that they do not form fit and simply hold a limb within the confines of a loosely fitting waterproof enclosure. Such products are marketed under the designation ShowerSafe waterproof cast protectors. Non-medicated stretch latex socks have also been provided as bandage protectors in order to keep the feet dry (DRI-Foot protectors). These are constructed to be reusable coverings for special circumstances rather than ultra thin disposable coverings for routine use.

### SUMMARY OF THE INVENTION

The present invention provides improved form-fitting disposable foot coverings which are usable in a wide variety of contexts for foot protection and the delivery of medicaments. Broadly, the foot coverings of the invention comprise a tubular body presenting a sole portion having an inner surface and an outer surface, and an uppers portion, where the body is configured to tightly fit over a person's foot with the inner surface of the sole region proximal to the person's sole and the uppers region extending upwardly and engaging the person's leg above the ankle. At a point of engagement an adhesive may be used to seal the foot covering against the person's body creating a watertight barrier to keep a desired portion of the user's body dry. At least the sole region of the covering (and preferably the entirety thereof) is formed of a thin, resilient, stretchable resin material. Also, medicament can be provided on at least a portion of the inner surface of the body, typically in patch or layer form on the inner surface of the sole portion. However, the medicament may be applied anywhere along the inner surface of the foot covering.

The foot coverings of the invention may be used for the protection of bare feet by preventing direct contact with contaminated surfaces, such as those found in locker rooms, shower rooms, airports, hospitals and nursing homes. An additional important use would be covering and protecting dressings or bandages on the foot or ankle of the wearer. The presence of medicament, and especially transdermal medicaments or topical ointments, assists in the administration thereof. Versions of the foot coverings can be essentially airtight, aiding in the elimination of dry feet; alternately, breathable versions having woven cloth or perforate uppers may be provided for extended wearing.

### BRIEF DESCRIPTION OF THE DRAWINGS

FIG. 1 is an elevational view with parts broken away of a foot covering in accordance with the invention, applied to a user's foot;

FIG. 2 is an elevational view similar to that of FIG. 1, but depicting another foot covering pursuant to the invention, disposed over a foot and ankle cast;

FIG. 3 is an elevational view illustrating another foot covering embodiment in accordance with the invention;

FIG. 4 is a sectional view taken along line 4-4 of FIG. 1 and depicting the cross section of the FIG. 1 foot covering;

FIG. 5 is a view similar to that of FIG. 4, but illustrating an embodiment including a padded, laminate sole portion;

FIG. 6 is a sectional view taken along line 6-6 of FIG. 3;

FIG. 7 is an elevational view of an embodiment in accordance with the invention, illustrating a foot covering having a perforate upper; and

FIG. 8 is an elevational view of a foot covering in accordance with the invention, applied to a user's foot.

FIG. 9 is an elevational view of a foot covering in accordance with the invention, applied to a user's foot.

### DETAILED DESCRIPTION OF THE PREFERRED EMBODIMENTS

Turning now to the drawings, a foot covering 10 in accordance with the invention is illustrated in FIGS. 1 and 4. Broadly speaking, the covering 10 is in the form of a tubular body 12 presenting a sole portion 14 and an uppers region 16; at least this uppers region of this embodiment is formed of a thin, resilient, stretchable synthetic or natural resin material. As shown, the cover 10 is designed to form-fit and closely conform to a person's foot, in the manner of a conventional sock.

In more detail, in the illustrated embodiment, the sole portion 14 which is proximal to the person's sole, presents an outer surface 18 and an opposed inner surface 20 (see FIG. 4). In accordance with one embodiment of the invention, at least a portion of the inner surface of the covering 10 is medicament coated or infused, e.g., the inner surface 20 supports a layer or patch of medicament 22. In order to improve traction, the outer surface 18 is fully or partially textured or treaded as at 26. The uppers region 16 is integral with the sole portion, and is of a length to extend over the person's ankle and terminates in an uppermost watertight bead 24.

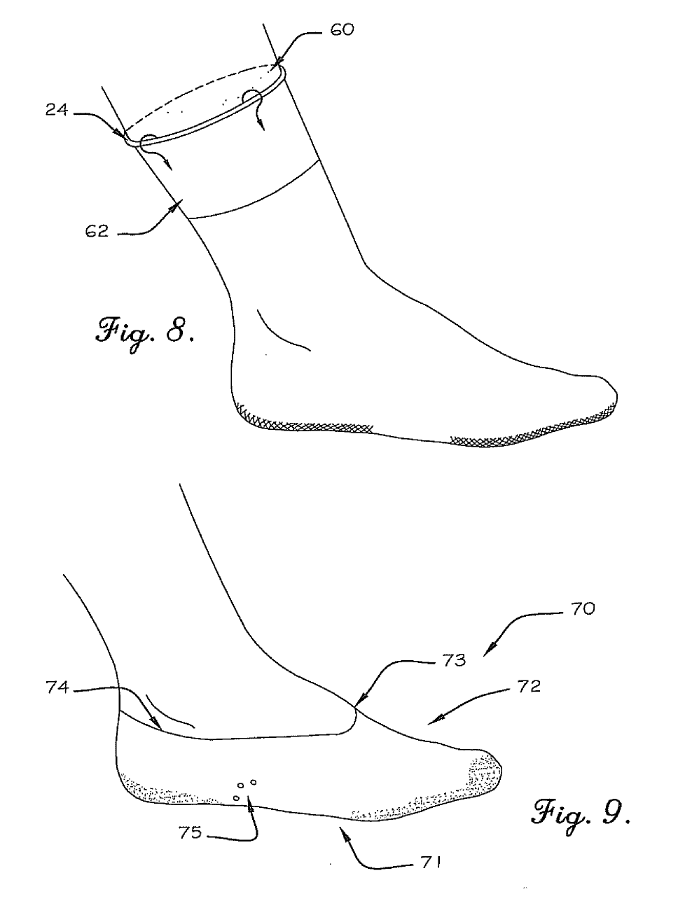

As shown in FIG. 8, in addition to watertight bead 24, or as an alternative to watertight bead 24, an adhesive 60 may be applied to the inner surface of the foot covering. The adhesive will ordinarily be applied in a band along the entire inner surface circumference of the foot covering. The adhesive is of a type that will adhere the foot covering to the body and create a watertight seal thereby keeping the foot covering in place and preventing water from entering beneath the foot covering. The exterior surface region 62 may have an adhesive resistant coating applied so that the user may roll down the foot covering from the top without the adhesive sticking to the exterior surface of the foot covering. Suitable adhesives are well known to those of ordinary skill in the art and may include but are not limited to medical grade hypo-allergenic pressure sensitive adhesives and medical grade acrylic pressure sensitive adhesives (collectively referred to as “PSAs”). When employed, the presence of the adhesive further enhances the water barrier properties of the foot covering.

When used in conjunction with a medicament that is coated onto the inner surface or infused into the material, the sock and medicament can be used to treat any and all cutaneous or systemic conditions. It is possible to use any medication that can be transmitted transdermally. For example, the medicament in conjunction with the sock may aid in the treatment of dermatitis by impregnating the sock with Lamisil or similar anti-fungal medicaments. It may also be used in the hydration of feet for those on dialysis. The present invention may also be used in the administration of antibiotics, hypertensive medications, to treat hypo-hydrosis with lanolin, to treat hyper-hydrosis with formaldehyde, to treat ulcerations on the feet which may occur as a result of complications caused by diabetes. Additionally, anti-yeast, vassal activators, medication used to control migraine headaches, and arthritis treatments may also be administered through use of the present invention. Free radicals of the foot cover can be used to bond the medications.

These and other topically and transdermally administered medications may all be employed as will be appreciated by those of ordinary skill in the art without the departing from the scope of the invention. The foot covering of the present invention can be manufactured with medications pre-infused into the sock. Thus, doctors could prescribe a medicament coated or infused sock that a user could then purchase from a pharmacy thereby avoiding the necessity of applying the medicament themselves. Medicament pre-coated or pre-infused socks would also be useful with non-prescription drugs.

The preferred resin materials useful in the invention are selected from the group consisting of latex, vinyl, polyurethane, polymer gel, and nitrile materials. Although the materials can have a variety of physical properties, generally the materials should have a tensile strength of from about 14 to 21 MPa and an ultimate elongation of from about 600 to 750% and a stress at 300% of from about 2 to 5 MPa. The material typically would have a relaxed thickness (i.e., when not worn) in the uppers region thereof of from about 0.001-0.010 inches, or preferably from about 0.002-0.005 inches. The materials contemplated for use in the present invention will have an elasticity and thickness such that they can be comfortably worn for long periods of time beneath the regular sock and shoe of the user or by themselves.

Again referring to FIGS. 1 and 4, it will be seen that the integrally formed covering 10 presents a sole portion 14 which is thicker than the upper region 16. This is to provide a more comfortable fit and padding for the user during walking. Such enhanced sole portion thickness should be from about 0.005 to 0.02 inches, more preferably from about 0.010 to 0.0175 inches. In addition, it is also within the ambit of the invention to provide a covering as illustrated in FIG. 5, having a sole portion 14a made up of two laminated resin materials, namely inner layer 28 and bottom layer 30. In this instance the layer 30 may be a soft, resilient synthetic resin gel to form the desired padding.

FIG. 1 also depicts a standard bandage 31 applied to the user's foot. Of course, given the stretchability and resilience of the body 10, such irregularities can readily be accommodated, i.e., the body 10 tightly fits over and form-fits around the bandage 31.

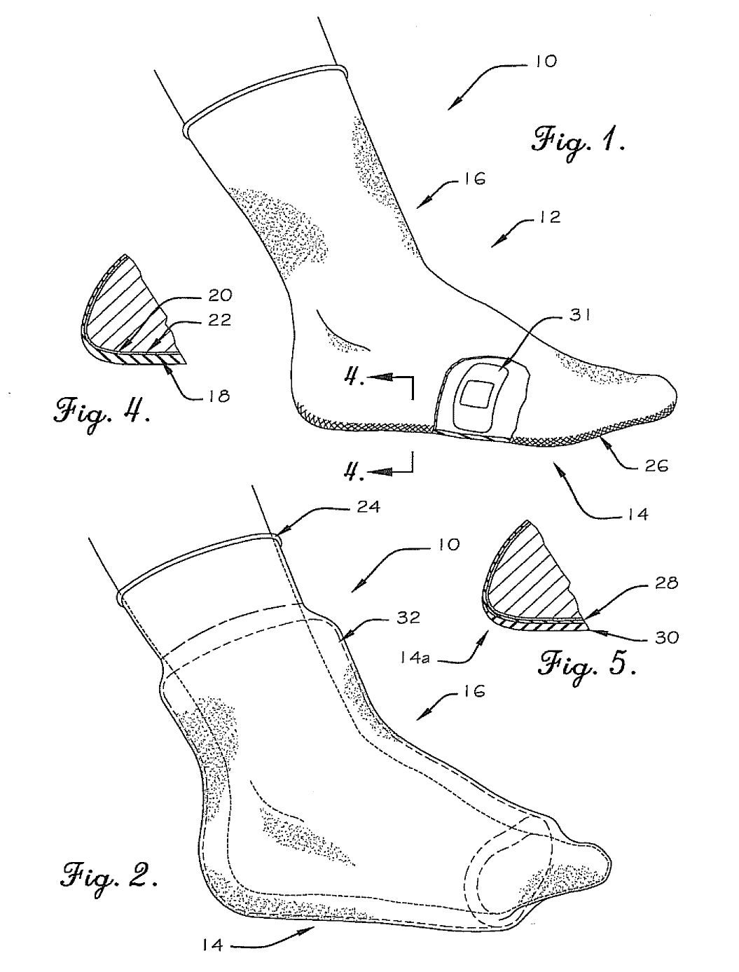

FIG. 2 illustrates the application of the cover 10 over a cast 32 applied to the ankle and foot of the user, The inherent stretchability and form-fit characteristics of the cover 10 allow the cover to adapt to various cast designs or large bandages.

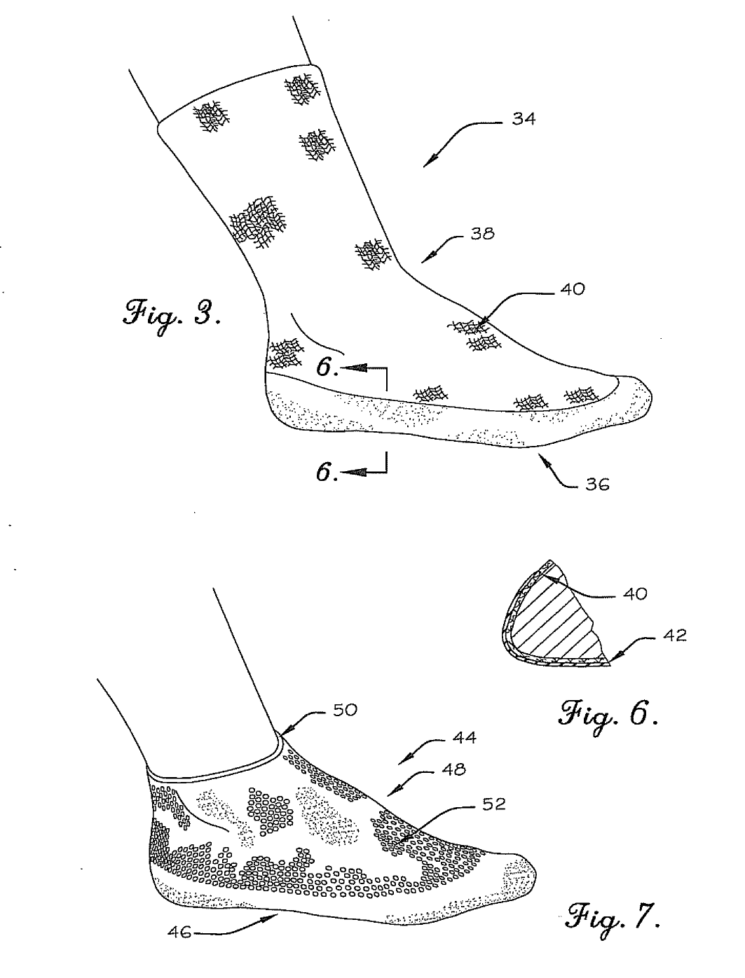

The invention may be utilized in other forms as depicted for example in FIG. 3. Here, a covering 34 is provided having a synthetic resin sole portion 36 and an upper 38 formed of woven cloth-like material 40. As shown in FIG. 6, the inner surface 42 of the sole portion 36 is lined with the woven material 40 and the latter is preferably secured to the inner surface 42. In this instance the medicament may be absorbed into the cloth lining. The sole portion 36 may be formed of any of the aforementioned resins and would have thicknesses similar to those described in connection with sole portion 14.

A further embodiment of the invention is illustrated in FIG. 7. Here, the covering 44 is entirely formed of resin material (i.e., either integrally from a single material or from separate materials) and has a sole portion 46 and an upper region 48. Here, the upper region 48 terminates in a bead 50 but is shorter in length than the region 16 described previously. However, in order to provide a cooler and more comfortable fit, the upper portion 48 is provided with a series of perforations or air holes 52. In embodiments where the foot covering does not extend above the user's ankle, water is more likely to get into the foot covering and perforations 52 or weep holes for drainage may be provided at different locations on the foot covering and in varying numbers and diameters as may be desired.

FIG. 9 illustrates the foot covering in a low rising form. Shown here, the covering 70 is entirely formed of resin material (i.e., either integrally from a single material or from separate materials) and has a sole portion 71 and upper portion 72. Here, the upper region rises, generally, mid-way up the instep 73 and rests below the ankle 74. Weep holes 75 may be placed in various locations for drainage, when used in a wet environment.

The various embodiments are of course suited for different uses or environments, as dictated by their specific design features. Additionally, the invention may be realized in a number of ways not specifically discussed herein, but within the spirit and scope of the invention.

1. A form-fitting disposable foot covering for the distribution and application of medicaments onto the skin of a wearer comprising a sock including a body presenting a heel portion shaped to receive the heel of a person, a toe portion shaped to receive the toes of a person, a sole portion having an inner surface and an outer surface, and an uppers portion, said body configured to conform to and tightly fit over a person's foot with the heel portion proximal to and fitting the heel of the person, the toe portion proximal to and fitting the toes of the person, the inner surface of the sole region proximal to the person's sole and the uppers region extending upwardly from the sole region and engaging the person's body, at least said sole heel and toe portions formed of a watertight, thin, resilient, stretchable resin material and having a medicament pre-applied on at least a portion of the inner surface of said body.

2. The foot covering of claim 1, said material selected from the group consisting of latex, vinyl, nitrile, and polymer gel materials.

3. The foot covering of claim 1, said material having a tensile strength of from about 14 to 21 MPa.

4. The foot covering of claim 1, said material having an ultimate elongation of from about 600 to 750%.

5. The foot covering of claim 1, said material having a stress at 300% of from about 2 to 5 MPa.

6. The foot covering of claim 1, the entirety of said covering being formed of said material.

7. The foot covering of claim 6, said material in the uppers region thereof having a thickness prior to application to a person's foot of from about 0.001-0.010 inches.

8. The foot covering of claim 7, said thickness being from about 0.002-0.005 inches.

9. The foot covering of claim 1, said heel, toe and sole portions having a thickness greater than the thickness of said uppers portion.

10. The foot covering of claim 9, said heel, toe and sole portions having a thickness of from about 0.005 to 0.02 inches.

11. The foot covering of claim 9, said covering being integrally formed of a single resin material.

12. The foot covering of claim 9, said sole portion being formed as a laminate of different kinds of resin material.

13. The foot covering of claim 12, said sole portion including a layer of resilient synthetic resin gel to form a padding.

14. The foot covering of claim 1, the exterior surface of said sole portion being textured for enhanced traction.

15. The foot covering of claim 1, said uppers portion formed of perforate resin material.

16. The foot coeverin of claim 1, said uppers poriton formed of wonven material.

17. The foot covering of claim 15, said woven material also secured to the inner surface of said sole portion.

18. The foot covering of claim 1, said medicament comprising a transdermal medicament.

19. The foot covering of claim 1, siad medicament comprising a topical medicament.

20. The foot covering of claim 1, the pre-applied medicament being pre-coated on at least a portion of the inner surface of said sole portion.

21. The foot covering of claim 1, wherein the medicament is infused into the material of the foot covering.

22. A form-fitting disposable foot covering comprising a tubular section having an inner surface and an outer surface, said tubular section configured to tightly fit over a person's foot with the inner surface proximal to the person's skin and an uppers region extending proximally from the foot and engaging the person's body formed of thin, resilient, strechable's resin material and having an adhesive for adhering the foot covering to the person's skin and creating a watertight seal between the foot covering and the person's skin.

23. The covering of claim 22, said material selected from the group consisting of latex, vinyl, nitrile, and polymer gel materials.

24. The covering of claim 22, said material having a tensile strength of from about 14 to 21 MPa.

25. The covering of claim 22, said material having an ultimate elongation of from about 600 to 750%.

26. The covering of claim 22, said material having a stress at 300% of from about 2 to 5 MPa.

27. The covering of claim 22, the entirety of said covering being formed of said material.

28. The covering of claim 27, said meterial in the uppers region thereof having a thickness prior to application to a person's extremity of from about 0.001-0.010 inches.

29. The covering of claim 28, said thickness being from about 0.002-0.005 inches.

30. The covering of claim 22, said covering being integrally formed of a single resin material.

31. The covering of claim 22, said uppers region formed of perforate resin material.

32. The covering of claim 22, the adhesive comprising a PSA.
